(19) United States
(12) Patent Application Publication
(10) Pub. No.: US 2007/0185571 A1
(43) Pub. Date: Aug. 9, 2007

(54) Apparatus and method for treating a regurgitant valve

(75) Inventors: Samir Kapadia, Orange, OH (US); Jay Yadav, Hunting Valley, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 11/700,295

(22) Filed: Jan. 31, 2007

Related U.S. Application Data
(60) Provisional application No. 60/765,666, filed on Feb. 6, 2006.

(51) Int. Cl. A61F 2/24

(52) U.S. Cl. 623/2.11; 623/2.17

(57) ABSTRACT

An apparatus for treating regurgitation of blood through a diseased valve having at least one leaflet includes a valve member having a support structure with a diameter and at least one valvular leaflet attached to the support structure. The valve member is dimensioned so that at least one leaflet of the diseased valve abuts at least one surface of the valve member to mitigate regurgitation of blood through the diseased valve. The apparatus further includes a suspending mechanism operatively coupled to the valve member. The suspending mechanism is configured so that the valve member is freely suspended within the diseased valve.

### RELATED APPLICATION

This application claims priority from U.S. provisional patent application Ser. No. 60/765,666, filed on Feb. 6, 2006, the subject matter of which is incorporated herein by reference.

### TECHNICAL FIELD

The present invention relates to an apparatus and method for treating and improving the function of dysfunctional heart valves. More particularly, the present invention relates to an apparatus and method that passively assists in closing the native valve leaflets to improve valve function of a regurgitant heart valve.

### BACKGROUND OF THE INVENTION

A heart valve may become defective or damaged from degeneration caused by congenital malformation, disease, and/or aging, etc. When the valve becomes defective or damaged, the leaflets may not function properly to effectively prevent blood flow when appropriate. For example, when a mitral valve functions properly, the mitral valve prevents regurgitation of blood from the left ventricle into the left atrium when the ventricle contracts. In order to withstand the substantial backpressure and prevent regurgitation of blood into the left atrium during the ventricular contraction, the chordae tendinae hold the anterior and posterior leaflets in place across the opening of the annular ring.

If the annulus of the mitral valve enlarges or dilates to a point where the attached leaflets are unable to fully close (malcoaptation) the opening, regurgitation may occur. Further, valve prolapse, or the forcing of the valve annulus and leaflets into the left atrium by backpressure in the left ventricle, may occur. Adverse clinical symptoms, such as chest pain, cardiac arrhythmias, dyspnea, may manifest in response to regurgitation or valve prolapse. As a result, surgical correction, either by valve repair procedures or by valve replacement, may be required.

Surgical reconstruction or repair procedures may include plication, chordal shortening, or chordal replacement. Another common repair procedure, known as annuloplasty, entails remodeling the valve annulus by implantation of a prosthetic ring to help stabilize the annulus and to correct or help prevent valve insufficiency. In situations where the valve leaflets exhibit lesions, reconstruction of one or more valve leaflets by securing grafts or patches to the leaflets, such as over lesions or holes formed in the leaflets, may be necessary. The repair or reconstruction of the leaflets is often done via an open-chest procedure, and can be complicated and time consuming.

### SUMMARY OF THE INVENTION

In one aspect of the present invention, an apparatus for treating regurgitation of blood through a diseased valve having at least one leaflet comprises a valve member having a supporting structure with a diameter and at least one valvular leaflet attached to the support structure. The valve member is dimensioned so that at least one leaflet of the diseased valve abuts at least one surface of the valve member to mitigate regurgitation of blood through the diseased valve. The apparatus further includes a suspending mechanism operatively coupled to the valve member. The suspending mechanism is configured so that the valve member is freely suspended within the diseased valve.

In another aspect of the present invention, a method is provided for treating regurgitation of blood through a diseased valve. One step of the method provides an apparatus comprising a valve member and a suspending mechanism operatively coupled to the valve member. The valve member further comprises a support structure and at least one valvular leaflet attached to the support structure. Next, a balloon is positioned in the diseased valve to determine the size and shape of the diseased valve. A valve member having a size and shape that corresponds to the size and shape of the diseased valve is then selected so that at least one leaflet of the valve coapts with the valve member. The apparatus is next introduced into a patient's vasculature and subsequently positioned in the diseased valve.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a perspective view of an apparatus for treating a regurgitant valve in accordance with the present invention;

FIG. 2 is a cross-sectional schematic view of a human heart;

FIG. 3A is a short-axis cross-sectional view of the human heart;

FIG. 3B is a partial short-axis cross-sectional view of the human heart;

FIG. 4A is a top view of a properly functioning mitral valve in an open position;

FIG. 4B is a top view of a properly functioning mitral valve in a closed position;

FIG. 4C is a top view of an improperly functioning mitral valve in a closed position;

FIG. 5A is a side view of a properly functioning mitral valve shown with its connection to the papillary muscles;

FIG. 5B is a side view of an improperly functioning mitral valve shown with its connection to the papillary muscles;

FIG. 6A is a schematic side view of an improperly functioning mitral valve during systole;

FIG. 6B is a schematic side view of the valve of FIG. 6A with a valve member implanted in the valve orifice;

FIG. 7A is a top view of the valve member in FIG. 1 showing a support structure comprised of an inflatable balloon (in a deflated configuration) that encircles the support structure;

FIG. 7B is a top view of the valve member in FIG. 7A showing the support structure in an inflated configuration;

FIG. 8 is a perspective view showing the apparatus in FIG. 1 with a helical-shaped anchoring portion;

FIG. 9 is a cross-sectional view showing a guidewire extending trans-septally through a human heart;

FIG. 10 is a cross-sectional view showing the guidewire extending through the mitral valve into the left ventricle;

FIG. 11 is a cross-sectional view showing a catheter advanced over the guidewire;

FIG. 12 is a cross-sectional view showing a deflated, two-layer balloon positioned within a distal end portion of the catheter;

FIG. 13A is a cross-sectional view of a two-layer inflatable balloon in an inflated configuration;

FIG. 13B is a cross-sectional view of the balloon shown in FIG. 13A in an ellipsoidal configuration;

FIG. 14 is a cross-sectional view showing the balloon of FIG. 13A in an inflated configuration positioned between the leaflets of the mitral valve;

FIG. 15 is a cross-sectional view showing the apparatus of FIG. 1 partly deployed in the left atrium;

FIG. 16 is a cross-sectional view of the apparatus of FIG. 1 deployed in the left atrium during diastole;

FIG. 17 is a cross-sectional view of the apparatus of FIG. 1 deployed in the left atrium during systole;

FIG. 18 is a cross-sectional view showing a guidewire extending through the inferior vena cava into the right atrium;

FIG. 19 is a cross-sectional view showing a catheter advanced over the guidewire;

FIG. 20 is a cross-sectional view showing an alternative embodiment of the apparatus in FIG. 1 partly deployed in the right atrium;

FIG. 21 is a cross-sectional view showing the apparatus of FIG. 20 deployed in the right atrium during diastole; and

FIG. 22 is a cross-sectional view showing the apparatus of FIG. 20 deployed in the right atrium during systole.

### DETAILED DESCRIPTION

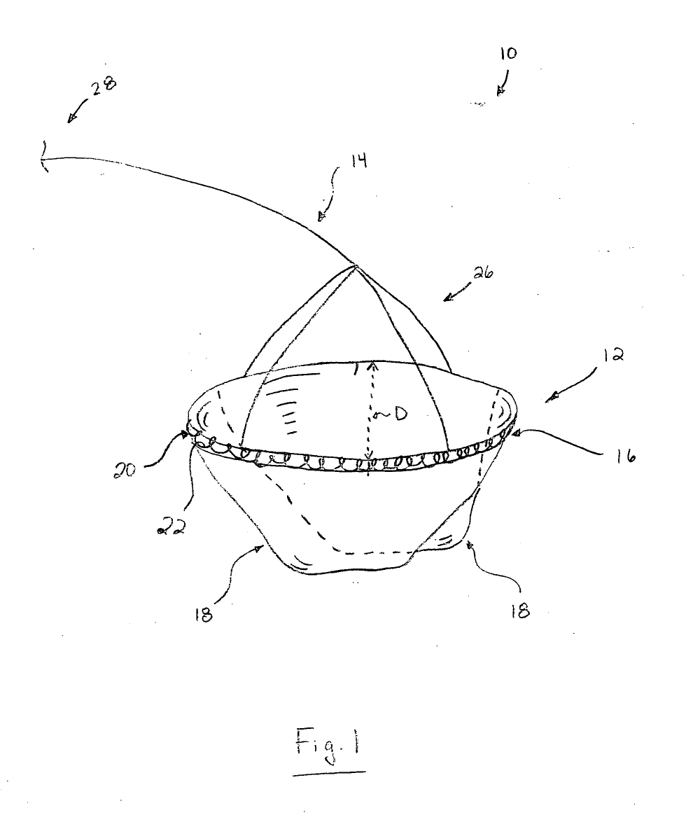

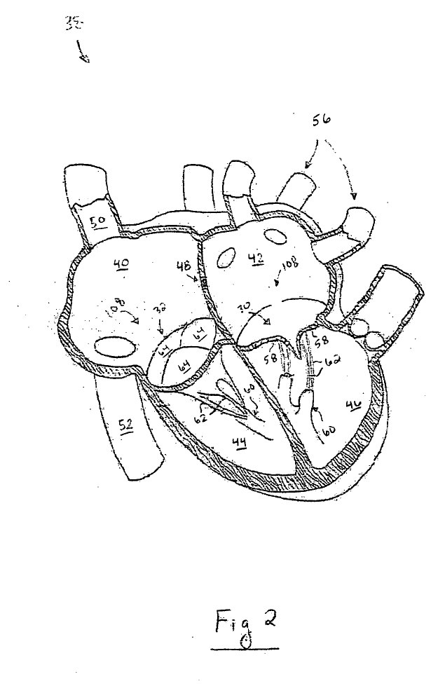

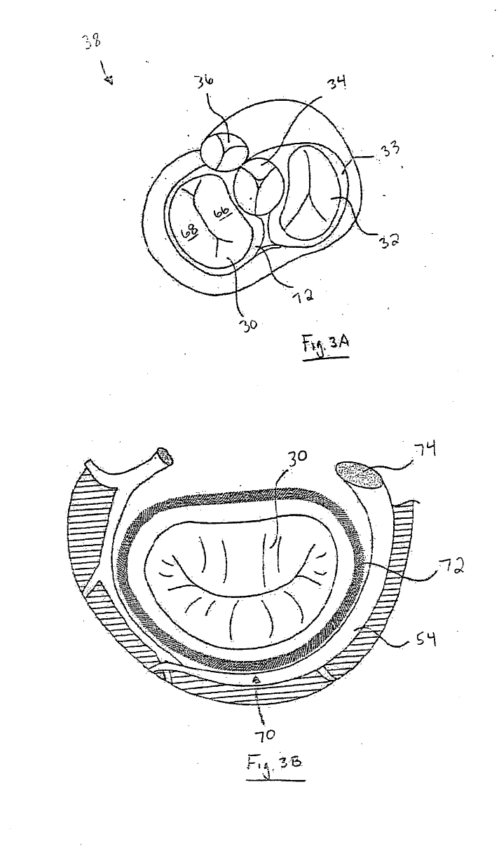

The present invention relates to an apparatus and method for treating and improving the function of dysfunctional heart valves. More particularly, the present invention relates to an apparatus and method that passively assists in closing the native leaflets to improve valve function of a regurgitant valve. As representative of the present invention, FIG. 1 illustrates an apparatus 10 for treating regurgitation of blood through a diseased valve having at least one leaflet. As described in further detail below, the present invention may be used to treat regurgitation of blood through atrioventricular valves, such as the mitral and tricuspid valves 30 and 32 (FIG. 2), and semilunar valves, such as the aortic and pulmonic valves 34 and 36 (FIG. 3A). Additionally or optionally, the present invention may be used to treat other diseased valves (not shown) of the arterial and venous vasculature.

FIG. 2 schematically illustrates a human heart 38 which includes four chambers: the right and left atria 40 and 42 and the right and left ventricles 44 and 46. The right and left atria 40 and 42 are divided by the interatrial septum 48. The thin-walled right atrium 40 receives deoxygenated blood from the superior vena cava 50, the inferior vena cava 52, and from the coronary sinus 54 (FIG. 3B). The thin-walled left atrium 42 (FIG. 2) receives oxygenated blood from pulmonary veins 56. The right and left ventricles 44 and 46 pump oxygenated and deoxygenated blood, respectively, throughout the body, and the pocket-like semilunar pulmonary valve 36 (FIG. 3A) and the aortic valve 34 prevent reflux into the ventricles. Atrial blood is pumped through the atrioventricular orifices, guarded by the tri-leaflet tricuspid valve 32 (FIG. 2) on the right side of the heart 38 and the bi-leaflet mitral valve 30 on the left side of the heart. The free edges of the leaflets 58 of the mitral valve 30 are attached to the papillary muscles 60 in the right and left ventricles 44 and 46 by chordae tendineae 62. Similarly, the free edges of the leaflets 64 of the tricuspid valve 32 are attached to the papillary muscles 60 in the right and left ventricles 44 and 46 by chordae tendineae 62.

FIG. 3A is a short-axis cross-sectional view of the heart 38 illustrating the mitral valve 30 in relation to the other valves of the heart; namely, the aortic valve 34, the tricuspid valve 32, and the pulmonary valve 36. The mitral valve 30 has two leaflets; an anterior leaflet 66 and a posterior leaflet 68. The anterior leaflet 66 is adjacent the aorta (not shown), and the posterior leaflet 68 is opposite the aorta. FIG. 3B is a partial short-axis cross-sectional view showing the mitral valve 30 in relation to the coronary sinus 54. The coronary sinus 54 wraps around a significant portion of the posterior aspect 70 of the mitral valve annulus 72. The ostium 74 of the coronary sinus 54 drains into the right atrium 40.

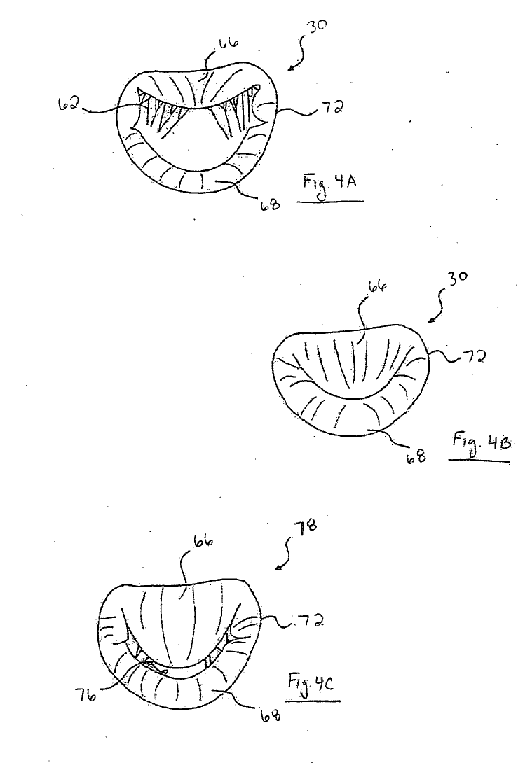

In FIGS. 4A and 4B, a top view of a properly functioning mitral valve 30 is shown. FIG. 4A shows the mitral valve 30 in its open position during diastole in which the posterior leaflet 68 is separated from the anterior leaflet 66. Portions of the chordae tendineae 62 can also be seen in FIG. 4A. FIG. 4B shows the properly functioning mitral valve 30 in the closed position during systole. In this figure, the anterior leaflet 66 and the posterior leaflet 68 contact one another and close the mitral valve 30 to prevent blood from flowing through the mitral valve from the left atrium 42 to the left ventricle 46.

FIG. 4C shows a top view of an improperly functioning mitral valve 30 in the “closed” position (i.e., during systole). In FIG. 4C, a regurgitant mitral valve orifice 76 is formed when the anterior leaflet 66 and the posterior leaflet 68 do not properly coapt. This may be caused by, for example, a dilatation of the annulus 72 caused by an enlargement of the left ventricle 46. As shown in FIG. 4C, this improper coaptation prevents the complete closure of the orifice 76 between the valve leaflets 58, thereby permitting blood to leak through the valve 30 from the left ventricle 46 to the left atrium 42 during systole. In other words, although the mitral valve 30 is in a contracted state, it is not actually closed so as to prevent blood flow therethrough since the leaflets 58 do not completely come together.

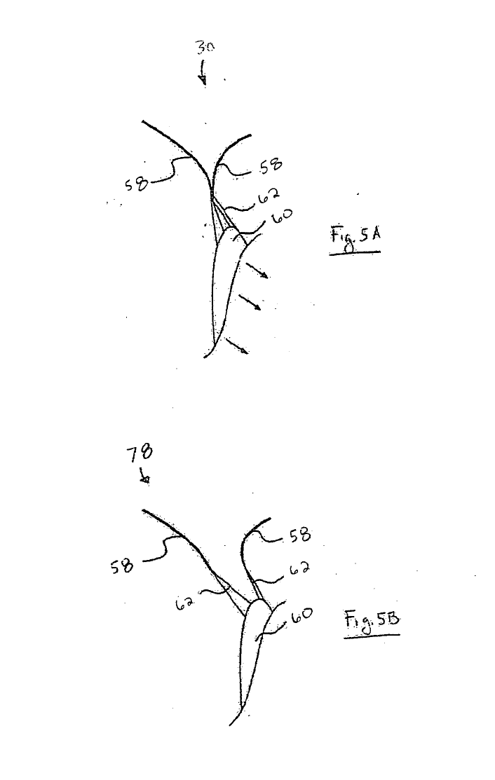

FIG. 5A shows a side view of a properly functioning mitral valve 30 in the closed position with the valve leaflets 58 properly coapted so as to prevent blood flow through the valve. The arrows in FIG. 5A show the movement of the papillary muscles 60 down and to the right resulting from such ventricle 46 dilatation. FIG. 5B shows a side view of an improperly functioning mitral valve 30 in which the valve leaflets 58 are not properly coapted due to, for example, dislocation of the papillary muscles 60. Such dislocation of the papillary muscles 60 may also be caused by enlargement of the left ventricle 46.

Such dysfunctioning valves, as shown in FIGS. 4C and 5B, may cause a reduction in forward stroke volume from the left ventricle 46. Also, a blood flow reversal into the pulmonary veins 56 may occur. Regurgitation of the mitral valve 30 may also arise from a combination of a dilated valve annulus 72 and dislocation of the papillary muscles 60.

As illustrated in FIG. 1, the present invention comprises a valve member 12 operatively coupled to a suspending mechanism 14. The valve member 12 can comprise an artificial valve. Different types of artificial heart valves are known in the art, including mechanical heart valves, bioprosthetic heart valves, and combinations thereof.

Mechanical heart valves are typically made from materials of synthetic origin like metals (e.g., stainless steel and molybdenum alloys), ceramics and polymers. Mechanical heart valves typically utilize a ball, a disc, valve leaflets or other mechanical valving devices to regulate the direction of blood flow through the prosthesis. Specific examples of mechanical heart valves are known in the art.

In addition to synthetic materials, materials of biological origin (e.g., bovine pericardial tissue, equine pericardial tissue, or bovine pericardial tissue) are typically used to construct bioprosthetic heart valves. Where the valve member 12 of the present invention comprises a bioprosthetic valve, the bioprosthetic valve may be made from one or more pieces of biological material formed into a mono-leaflet or multi-leaflet conduit having dimensions that correspond to the dimensions of the native valve. Specific examples of bioprosthetic valves are known in the art.

As for biological materials for use with the valve member 12, a variety of fixed tissues may be used, including, for example, pericardium, peritoneum, facia mater, dura mater, and vascular tissues. Tissues may be fixed with a variety of chemical additives, such as aldehydes and epoxies, for example, so as to render them non-immunogenic and biologically stable. Engineered tissues may also be used with the valve member 12. Tissue substrates may be constructed from a variety of materials, such as resorbable polymers (e.g., polylactic acid, polyglycolic acid, or collagen). These substrates may then be coated with biologically active molecules to encourage cellular colonization. Additionally, these tissues may be constructed in vitro, for example, using the patient's own cells or using universal cell lines. In this way, the tissue may maintain an ability to repair itself or grow with the patient.

The biological materials may also be subjected to surface modification techniques to make them selectively bioreactive or non-reactive. Such modification may include physical modification, such as texturing with surface coatings (e.g., hydrophilic polymers) and ceramics (e.g., pyrolytic carbon, zirconium nitrate, and aluminum oxide). Other types of modifications may include electrical modification, such as ionic modification, and coating with biologically derived coatings, such as heparin, albumin, and a variety of growth healing modification factors (e.g., vascular endothelial growth factors or cytokines).

The valve member 12 of the present invention assists in closing a diseased valve to prevent regurgitation by increasing the coaptation area of the valve leaflets and/or decreasing the coaptation depth of the valve leaflets during systole. Where the apparatus 10 is used to treat a diseased mitral valve 78, for example, increasing coaptation of the diseased mitral valve is generally accomplished by placing the valve member 12 in the regurgitant mitral valve orifice 76, thereby providing a surface against which the mitral valve leaflets 58 may abut (i.e., coapt) in order to close the mitral valve during systole. The valve member 12 assists in substantially closing the diseased mitral valve 78 without altering the shape of the valve annulus 72 and/or repositioning the papillary muscles 60. Further, because the valve member 12 comprises an artificial valve, blood flow is essentially unimpeded through the diseased valve during diastole.

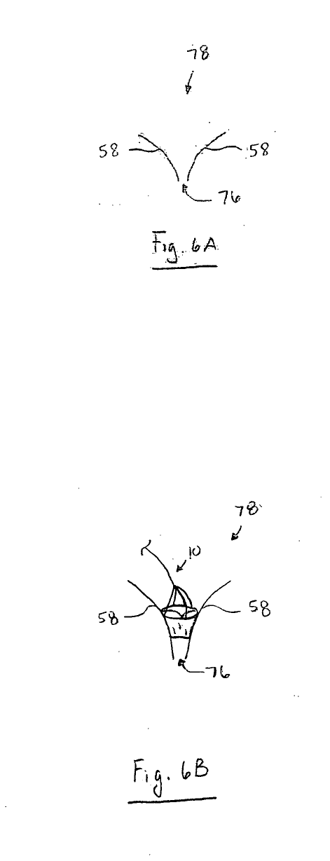

FIG. 6A illustrates a schematic side view of the leaflets 58 of a dysfunctional mitral valve 78 during systole. As seen in FIG. 6A, the leaflets 58 do not coapt so as to close the regurgitant mitral valve orifice 76. Therefore, regurgitant blood flow will occur through the mitral valve 78 during systole. FIG. 6B illustrates the valve 78 of FIG. 6A during systole with the valve member 12 implanted in the regurgitant mitral valve orifice 76. As can be seen, the presence of the valve member 12 will block regurgitant blood flow through the mitral valve 78 during systole as the anterior and posterior leaflets 66 and 68 abut against the surface of the valve member. In other words, the valve member 12 “plugs” the regurgitant mitral valve orifice 76 during systole to hinder or prevent blood from leaking through the valve 78.

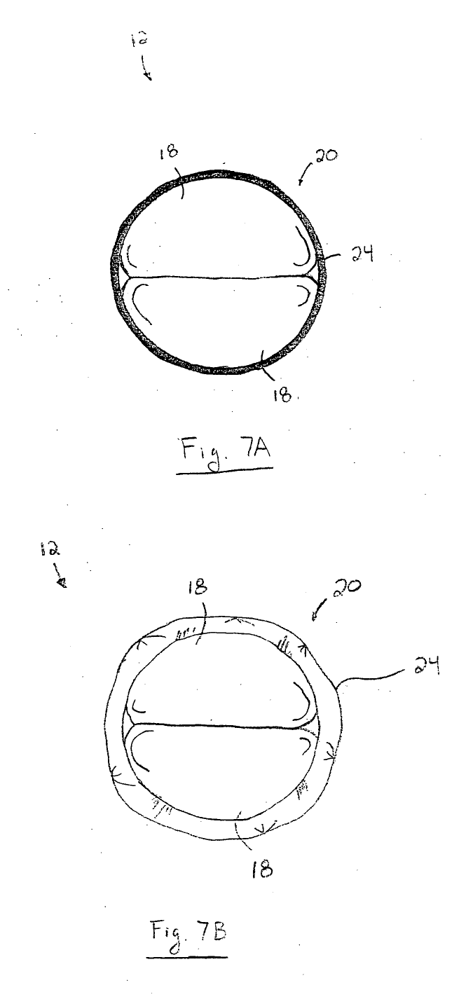

As shown in FIGS. 1, 7A and 7B, the valve member 12 further comprises a collapsible support structure 16 having a diameter D and at least one valvular leaflet 18 attached to the support structure. The valvular leaflet(s) 18 may be attached to the support structure 16 via sutures, staples, pins, adhesives, or the like. The support structure 16 further comprises an adjustable sizing member 20 for adjusting the position of the valve member 12 within a diseased valve. The adjustable sizing member 20 may be integrally disposed within the support structure 16 or, alternatively, fluidly connected to the support structure.

As shown in FIG. 1, the adjustable sizing member 20 may comprise a flexible ring 22 made of a metal or metal alloy, such as Nitinol, that encircles the entire support structure 16. Alternatively, the adjustable sizing member 20 may only encircle a portion, such as one-half or three-quarters, of the support structure 16. Where the adjustable sizing member 20 comprises a flexible ring 22, the flexible ring may be adjusted to increase or decrease the diameter D of the support structure 16. For example, the flexible ring 22 may be tensioned via an actuatable mechanism (not shown; described further below) so as to decrease the diameter D of the support structure 16.

In addition to a flexible ring 22, the adjustable sizing member 20 may also comprise an inflatable ring 24 as shown in FIGS. 7A and 7B. The inflatable ring 24 may encircle the entire support structure 16 or, alternatively, only a portion of the support structure. The inflatable ring 24 may have a deflated configuration (FIG. 7A) or a deflated configuration (FIG. 7B). The inflatable ring 24 may be inflated or deflated as needed to adjust the diameter D of the support structure 16. To decrease the diameter D of the support structure 16, for example, the inflatable ring 24 may be inflated as shown in FIG. 7B.

To adjust the configuration of the adjustable sizing member 20, the apparatus 10 may also comprise an actuatable mechanism. The actuatable mechanism may include, for example, a pressure-sensitive switch capable of causing the adjustable sizing member 20 to change configuration during the cardiac cycle. During systole, for example, the pressure-sensitive switch may cause the adjustable sizing member 20 to decrease in size and, in turn, cause the diameter D of the support structure 16 to decrease. Alternatively, the actuatable mechanism may also include a wire or cable operatively connected to the adjustable sizing member 20. The wire or cable may be selectively tensioned, for example, so that the diameter D of the support structure 16 is decreased.

The suspending mechanism 14 of the present invention may have a variety of configurations, such as the wire-like configuration shown in FIG. 1, and may also have a rigid, semi-rigid, or flexible shape. Where the suspending mechanism 14 has a wire-like configuration, the suspending mechanism may be constructed of either monofilament or multifilament constructions, such as braids or cables, for example. The suspending mechanism 14 may be made from a biocompatible material or may otherwise be treated with a material or combination of materials to impart biocompatability. Materials such as high strength polymers, including liquid crystal polymers and ultra high molecular weight polyethylene fibers may be suitable to provide desirable mechanical and fatigue properties. Suitable metals may include stainless steel, titanium alloys, and cobalt-chrome alloys, for example.

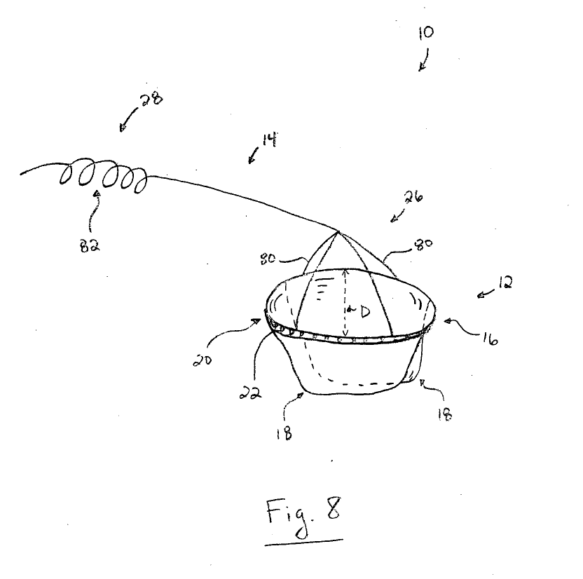

As illustrated in FIG. 8, the suspending mechanism 14 includes a distal end portion 26 and a proximal end portion 28. The distal end portion 26 is operatively connected to the valve member 12. Where the suspending mechanism 14 has a wire-like configuration (FIG. 8), the distal end portion 26 may comprise at least one support member 80 capable of being securely attached to the valve member 12. As illustrated in FIG. 8, for example, the distal end portion 26 of the suspending mechanism 14 includes four wire-like support members 80 securely attached to the valve member 12.

The proximal end portion 28 of the support mechanism 14 further includes an anchoring portion 82 capable of securing the apparatus 10 to a desired location in a patient's vasculature. For example, the anchoring portion 82 may be secured to a vascular structure, such as a wall of the left atrium 42. Alternatively, the anchoring portion 82 may be secured to a vessel wall, such as a wall of the superior or inferior vena cava 50 and 52. The anchoring portion 82 may have a variety of configurations, including the spiral or helical-shaped configuration shown in FIG. 8. The anchoring portion 82 may also comprise a septal occluder (not shown), such as the AMPLATZER® septal occluder, available from AGA Medical Corporation, located in Golden Valley, Minn.

The suspending mechanism 14 serves to securely anchor the apparatus 10 in a desired location, and ensure that the valve member 12 is freely suspended within a diseased valve. By “freely suspended” it is meant that the valve member 12 hangs or dangles in the diseased valve and, importantly, is not attached or anchored to the diseased valve during the cardiac cycle. In other words, the suspending mechanism 14 ensures that the valve member 12 contacts a portion of the diseased valve, such as a leaflet, during systole and then, during diastole, does not contact the diseased valve.

To facilitate positioning of the apparatus 10 in a diseased valve, the apparatus may include at least one radiographically opaque marking (not shown). The radiographically opaque marking may be located at the valve member 12 or, alternatively, at any other portion of the apparatus 10. The radiographically opaque marking can be any one or combination of materials or devices with significant opacity. Examples of such radiographically opaque markings include, but are not limited to, a steel mandrel sufficiently thick to be visible on fluoroscopy, a tantalumlpolyurethane tip, a gold-plated tip, bands of platinum, stainless steel or gold, soldered spots of gold, and polymeric materials with a radiographically opaque filter such as barium sulfate.

The particular position selected to implant the valve member 12 may depend on a variety of factors, such as the condition of the patient's heart 38, including the valve leaflets, the delivery technique utilized to implant the apparatus 10, the type of valve member utilized to treat the valve, and other similar factors. Particular positions may be selected based on factors such as the geometry, including size and shape, of the native valve. For instance, the valve member 12 may be configured to be positioned between the mitral valve leaflets 58, below the free ends of the valve leaflets, or at a level of the valve annulus 72 so that the valve member permits the valve 78 to close during systole and thus prevent regurgitant blood flow from occurring.

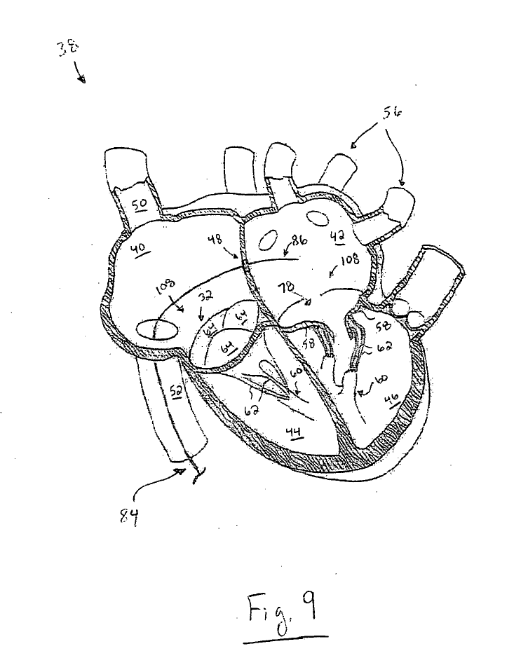

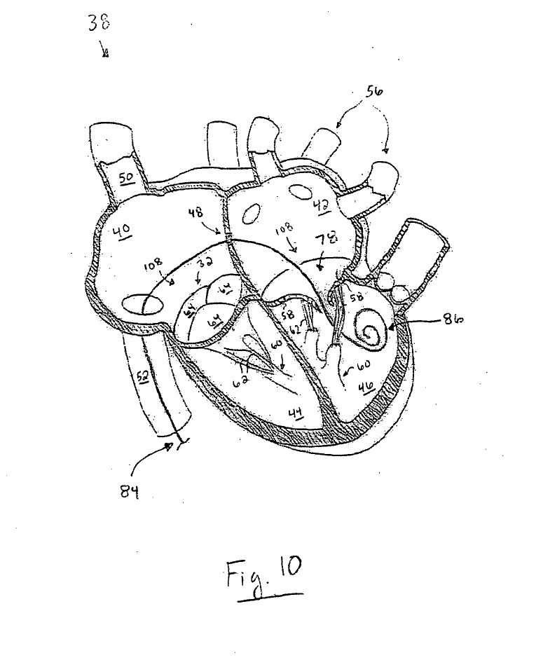

To treat regurgitation of blood through a diseased heart valve 108, such as a diseased mitral valve 78, the present invention may be percutaneously delivered to the left atrium 42 as illustrated in FIGS. 9-17. A guidewire 84 is inserted into a patient's vasculature via a femoral vein (not shown) or jugular vein (not shown) and, under image guidance (e.g., fluoroscopy, ultrasound, magnetic resonance, computed tomography, or combinations thereof), respectively steered through the patient's vasculature into the inferior vena cava 52 or superior vena cava 50. The guidewire 84 is then passed across the right atrium 40 so that the distal end 86 of the guidewire pierces the interatrial septum 48 as shown in FIG. 9. The guidewire 84 is extended across the left atrium 42 and then downward through the diseased mitral valve 78 so that the distal end 86 of the guidewire is securely positioned in the left ventricle 46 (FIG. 10).

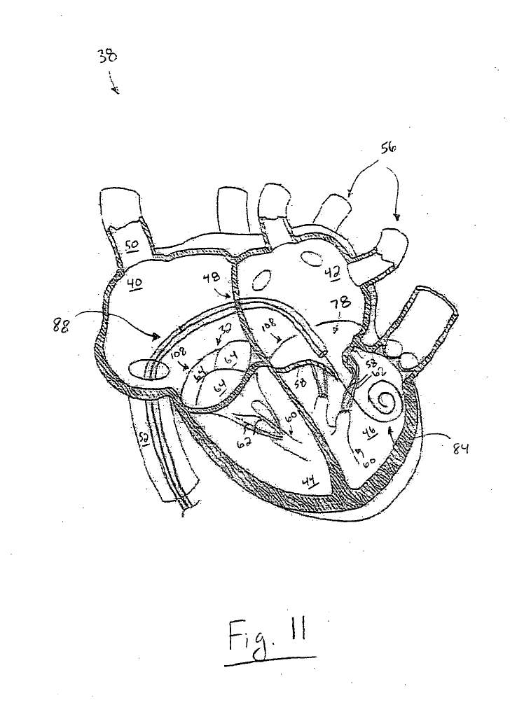

After the guidewire 84 is appropriately positioned in the patient's heart 38, a catheter 88 is passed over the guidewire as shown in FIG. 11. The catheter 88 may be comprised of a flexible, resiliently yieldable material such as silicone, PTFE, ePTFE, plastic polymer, or the like.

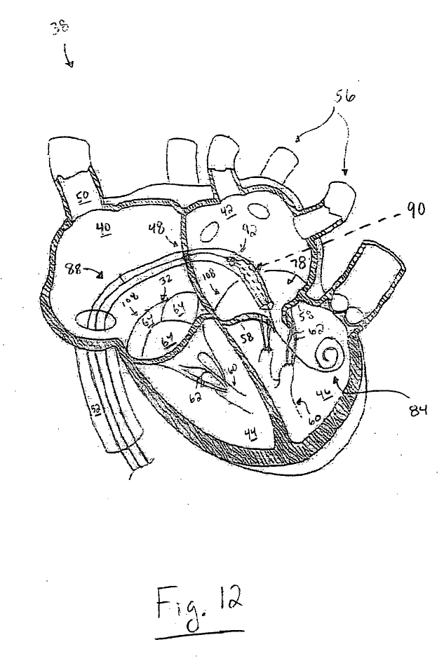

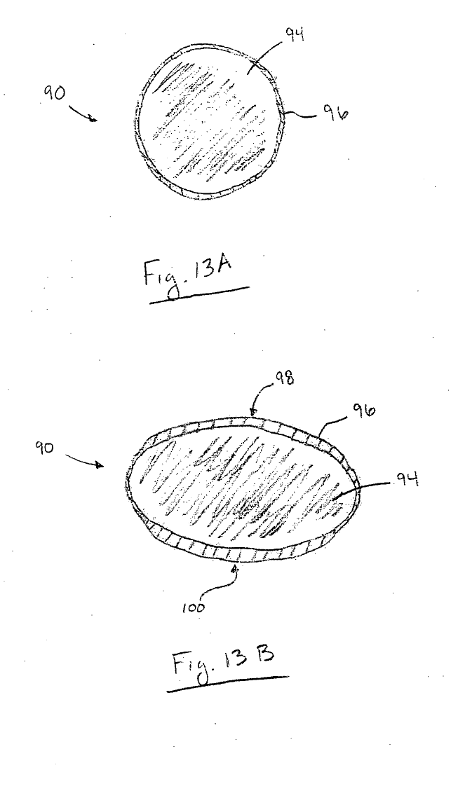

An inflatable balloon 90 is next attached at the proximal end (not shown) of the guidewire 84 in a deflated configuration, and then advanced over the guidewire until the balloon is positioned within the distal end portion 92 of the catheter 88 (FIG. 12). The balloon 90 is used to measure the geometry of the regurgitant mitral valve orifice 76 and, as shown in FIG. 13A, has a two-layer configuration. The first layer 94 can be made from a conventional material, such as PTFE, elastomeric materials including latex, silicone, polyolefin copolymers, or any other suitable balloon materials known in the art.

The second layer 96 may be made of a woven or braided cloth such as nylon, silk, gauze, ePTFE, or the like. The second layer 96 may have a uniform thickness and may fully or partially encapsulate the first layer 94. Alternatively, the second layer 96 may have different sections of varying thickness. As shown in FIG. 13B, for example, the anterior and posterior sections 98 and 100 of the second layer 96 may be thicker than other sections of the second layer. As a consequence, the thicker sections impart a greater resistance to the first layer 94 when the balloon 90 is inflated and, as illustrated in FIG. 13B, cause the balloon to obtain an ellipsoidal or crescent-like shape.

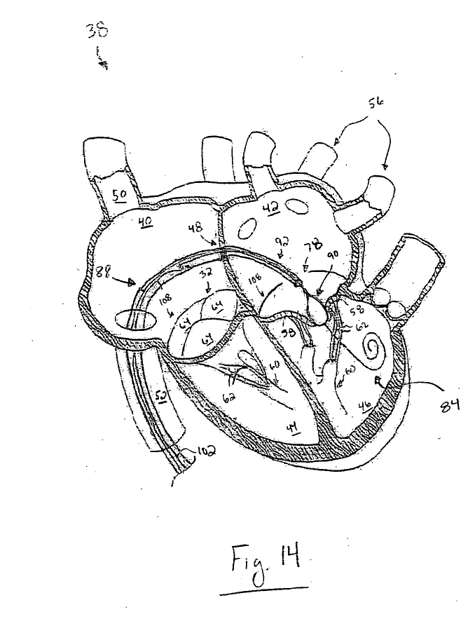

Once the balloon 90, in a deflated configuration, is positioned within the distal end portion 92 of the catheter 88, the catheter is then manipulated so that the balloon is progressively freed from the catheter. As shown in FIG. 14, the balloon 90 is then positioned in the regurgitant mitral valve orifice 76 and inflated so that at least one leaflet 58 of the diseased mitral valve 78 coapts with at least one surface of the balloon. Coaptation of the valve leaflets 58 may be monitored by any image-based means. Where the balloon 90 has opacity, for example, magnetic resonance imaging (MRI) or computed tomography (CT) may be used to monitor the extent of coaptation between the leaflets 58 and the balloon.

Additionally, the amount of regurgitation through the diseased mitral valve 78 may be monitored via an echocardiographic technique (e.g., transesophageal echocardiography, doppler echocardiography, 2-D echocardiography, and/or color echocardiography). When regurgitation has been sufficiently or entirely prevented, the geometry of the balloon 90 is then measured by, for example, determining the diameter of the balloon in a plurality of dimensions. Additionally or optionally, the distance between the balloon 90 and the interatrial septum 48 may be measured by MRI, CT, ultrasound, fluoroscopy, or other similar technique.

After determining the geometry of the balloon 90, the balloon is deflated and removed from the patient's vasculature. Based upon the previously measured dimensions of the balloon 90, an appropriately-sized apparatus 10 is then selected. For instance, the selected apparatus 10 will have a valve member 12 whose geometry corresponds to the measured geometry of the balloon 90. Additionally, where the distance between the balloon 90 and the interatrial septum 48 was measured, the suspending mechanism 14 of the apparatus 10 will also have the corresponding length.

Once the appropriately-sized apparatus 10 is selected, the apparatus is then attached to the proximal end (not shown) of the guidewire 84. A positioning wire 102 or other similar device useful for advancing the apparatus 10 over the guidewire 84 is then attached to the proximal end portion 28 of the suspending mechanism 14. An axial force is applied to the positioning wire 102 so that the apparatus 10 is passed over the guidewire 84 and positioned at the distal end portion 92 of the catheter 88.

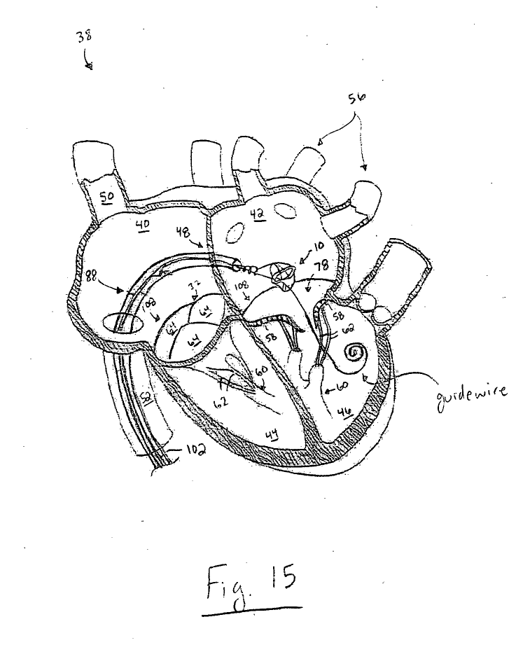

Upon reaching the distal end portion 92 of the catheter 88, the apparatus 10 is progressively freed from the catheter as shown in FIG. 15. As the apparatus 10 is progressively freed from the catheter 88, the position of the apparatus in the left atrium 42 can be monitored, controlled, and/or quality assured by imaging systems of various kinds. For example, X-ray machines, fluoroscopic machines, ultrasound, CT, MRI, positron emission tomography (PET), and other imaging devices may be used.

The apparatus 10 is next appropriately positioned in the left atrium 42 after being freed from the catheter 88. For instance, where the suspending mechanism 14 is configured as shown in FIG. 8, the anchoring portion 82 is urged toward the interatrial septum 48 until the anchoring portion contacts the interatrial septum. The anchoring portion 82 is then manipulated so that the anchoring portion is securely positioned about the interatrial septum 48. Alternatively, where the anchoring portion 82 comprises a septal occluder, the anchoring portion may engage the interatrial septum 48 so that the septal occluder straddles or braces the interatrial septum and thereby securely anchors the apparatus 10 in the left atrium 42.

After the apparatus 10 is secured in the left atrium 42, the configuration of the valve member 12 may be adjusted as needed. For example, the diameter D of the support structure 16 may be increased or decreased so that the valve member 12 may be freely suspended in the regurgitant mitral valve orifice 76. Where the adjustable sizing member 20 comprises an inflatable ring 24 as shown in FIGS. 7A and 7B, the inflatable ring may be inflated to facilitate coaptation of the mitral valve leaflets 58 during systole. If the valve leaflets 58 contact the valve member 12 during diastole, however, then the inflatable ring 24 may be selectively deflated so that the valve leaflets no longer coapt with the valve member during diastole.

The position of the valve member 12 may also be adjusted after the apparatus 10 is secured in the left atrium 42. For example, where the anchoring portion 82 of the suspending mechanism 14 comprises the helical or spiral-shaped configuration shown in FIG. 8, the suspending mechanism may be rotated in a clockwise or counter-clockwise manner so that the valve member 12 is respectively advanced or retracted within the regurgitant mitral valve orifice 76. Additionally or optionally, the position of the valve member 12 may be adjusted by cinching or bending the suspending mechanism 14.

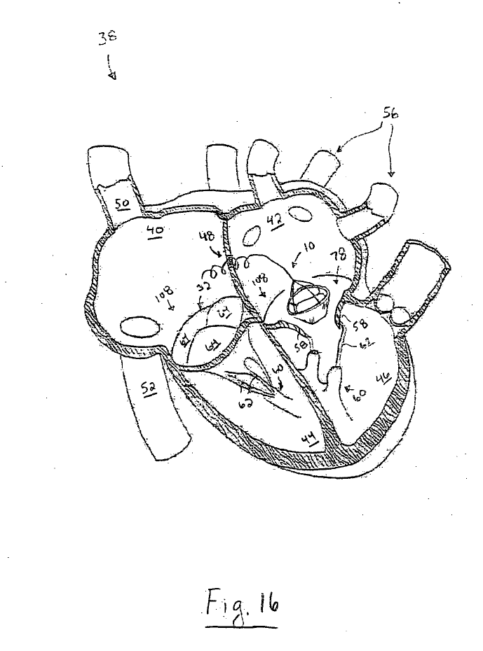

Depending upon the location and geometry of the regurgitant mitral valve orifice 76, the valve member 12 may be suspended at any one of a number of different positions within the diseased mitral valve 78. As illustrated in FIG. 16, for example, the valve member 12 may be positioned approximately level to the mitral valve annulus 72. Alternatively, at least a portion of the valve member 12 may be positioned below the free ends of the mitral valve leaflets 58.

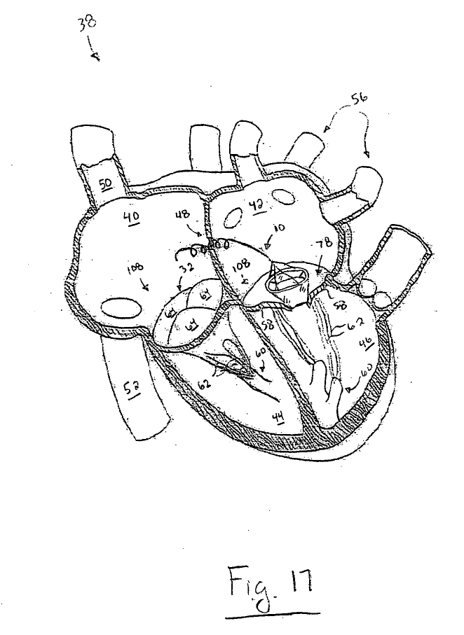

After the apparatus 10 is appropriately positioned in the left atrium 42, the positioning wire 102 is disconnected from the apparatus and, along with the guidewire 84, withdrawn from the patient's vasculature. With the valve member 12 freely suspended in the diseased mitral valve 78, blood may flow normally through and around the valve member during diastole (FIG. 16). Then, during systole, at least one leaflet 58 of the diseased mitral valve 78 can coapt with a surface of the valve member 12 as shown in FIG. 17. In doing so, the leaflet(s) 58 abut the valve member 12 and buttress the diseased mitral valve 78 so that regurgitant blood flow is substantially reduced or eliminated.

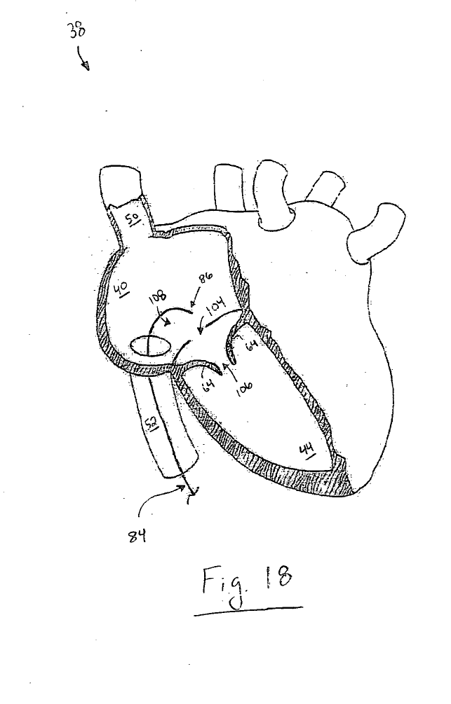

In an alternative embodiment of the present invention, the apparatus 10 may be used to reduce or eliminate regurgitant blood flow through a diseased tricuspid valve 104. The apparatus 10 shown in FIGS. 18-22 is identically constructed as the apparatus shown in FIG. 1, except where as described below.

As shown in FIGS. 18-22, a percutaneous approach may be used to deliver the apparatus 10 to the diseased tricuspid valve 104. A guidewire 84 may be inserted into a patient's femoral vein (not shown) or jugular vein (not shown) and, under image guidance (e.g., fluoroscopy, ultrasound, MRI, CT, or combinations thereof, respectively steered through the inferior vena cava or superior vena cava 52 and 50 into the right atrium 40 (FIG. 18).

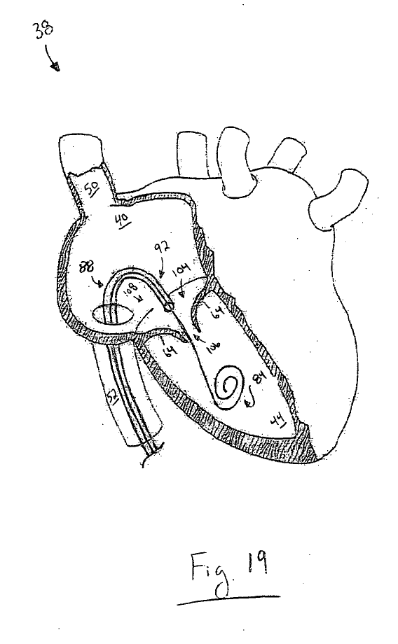

Once the distal end 86 of the guidewire 84 has reached the right atrium 40, the distal end may be hinged downward toward the diseased tricuspid valve 104. The guidewire 84 may then be urged through the diseased tricuspid valve 104 so that the distal end 86 enters the right ventricle 44. The guidewire 84 may next be positioned in the right ventricle 44 so that the guidewire is securely positioned within the inferior vena cava 52, the right atrium 40, and the right ventricle 44 (FIG. 19).

After the guidewire 84 is secured in the patient's heart 38, a catheter 88 may be passed over the guidewire and advanced into the right atrium 40. The inflatable balloon 90 (FIG. 13A) may next be attached at the proximal end (not shown) of the guidewire 84 in a collapsed configuration, and then advanced over the guidewire until the balloon is positioned within the distal end portion 92 of the catheter 88. Once the balloon 90 is positioned at the distal end portion 92, the catheter 88 can be manipulated so that the balloon is progressively freed from the catheter. The balloon 90 may then be positioned in a regurgitant tricuspid valve orifice 106 and inflated so that at least one leaflet 64 of the diseased tricuspid valve 104 coapts with at least one surface of the balloon.

Coaptation of the valve leaflets 64 with the surface of the balloon 90 may be monitored by any image-based means. Where the balloon 90 has opacity, for example, MRI or CT may be used to monitor the degree of coaptation between the leaflets and the balloon. Additionally, the amount of regurgitation through the diseased tricuspid valve 104 may be monitored via an echocardiographic technique (e.g., transesophageal echocardiography, doppler echocardiography, 2-D echocardiography, and/or color echocardiography). When regurgitation has been sufficiently or entirely prevented, the geometry of the balloon 90 may then be measured by, for example, determining the diameter of the balloon in a plurality of dimensions. Additionally or optionally, the distance between the balloon 90 and the inferior vena cava 52 may be measured by MRI, CT, ultrasound, fluoroscopy, or other similar technique.

After determining the geometry of the balloon 90, the balloon may be deflated and removed from the patient's vasculature. Based on the previously measured dimensions of the balloon 90, an appropriately-sized apparatus 10 may then be selected. For instance, the selected apparatus 10 may have a valve member 12 whose geometry corresponds to the measured geometry of the balloon 90. Additionally, where the distance between the balloon 90 and the inferior vena cava 52 was measured, the suspending mechanism 14 of the apparatus 10 may have the corresponding length.

Once an appropriately-sized apparatus 10 is selected, the apparatus may then attached to the proximal end of the guidewire 84. A positioning wire 102 or other similar device useful for advancing the apparatus 10 over the guidewire 84 may be operatively attached to the proximal end portion 28 of the apparatus. An axial force can then applied to the positioning wire 102 so that the apparatus 10 is passed over the guidewire 84. The apparatus 10 may then be advanced along the guidewire 84 until the apparatus reaches the distal end portion 92 of the catheter 88.

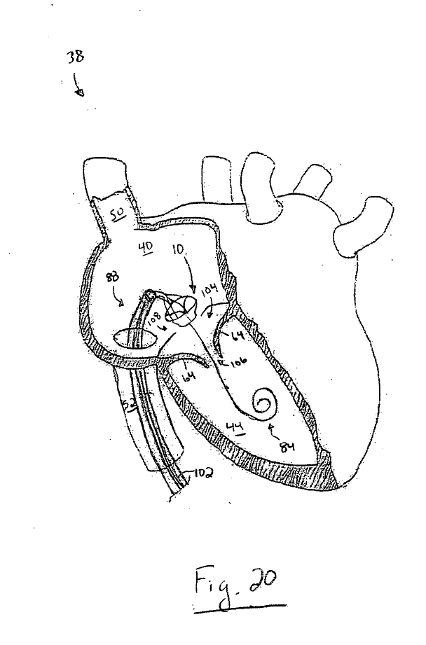

Upon reaching the distal end portion 92 of the catheter 88, the apparatus 10 may be progressively freed from the catheter as shown in FIG. 20. As the apparatus 10 is progressively freed from the catheter 88, the position of the apparatus within the right atrium 40 can be monitored, controlled, and/or quality assured by imaging systems of various kinds. For example, X-ray machines, fluoroscopic machines, ultrasound, CT, MRI, PET, and other imaging devices may be used.

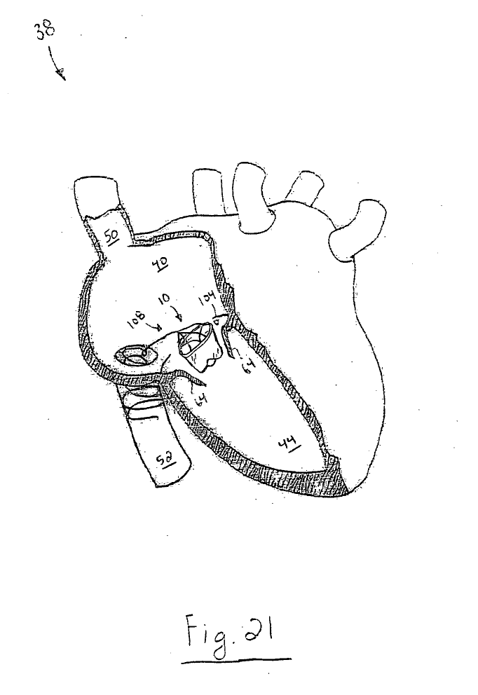

Once the apparatus 10 is freed from the catheter 88, the apparatus may be secured in the right atrium 40 by appropriately positioning the suspending mechanism 14 in the inferior vena cava 52. As shown in FIG. 21, for example, the anchoring portion 82 may be positioned within a portion of the inferior vena cava 52. The anchoring portion 82 may alternatively be placed in a portion of the superior vena cava 50.

After securing the apparatus 10 in the right atrium 40, the configuration of the valve member 12 may be adjusted so that the valve member is freely suspended in the regurgitant tricuspid valve orifice 106. Where the adjustable sizing member 20 comprises a flexible ring 22 as shown in FIG. 1, the configuration of the valve member 12 may be adjusted as needed. For example, the actuatable mechanism may be used to tension the support structure 16 so that the diameter D of the valve member 12 is decreased.

The position of the valve member 12 may also be adjusted by rotating or twisting the anchoring portion 82 in a clockwise or counter-clockwise manner so that the valve member is respectively advanced or retracted within the regurgitant tricuspid valve orifice 106. Alternatively, the position of the valve member 12 may be adjusted by bending or cinching the suspending wire 14. By adjusting the position of the valve member 12, at least one leaflet 64 of the diseased tricuspid valve 104 will coapt with the valve member during systole and, during diastole, the valve member will not contact the diseased tricuspid valve.

Depending upon the location and geometry of the regurgitant tricuspid valve orifice 106, the valve member 12 may be freely suspended at any one of a number of different positions. As illustrated in FIG. 21, for example, the valve member 12 may be positioned approximately level to the annulus 33 of the valve 104. Alternatively, the valve member 12 may be positioned so that at least a portion of the valve member is positioned below the free ends of the tricuspid valve leaflets 64.

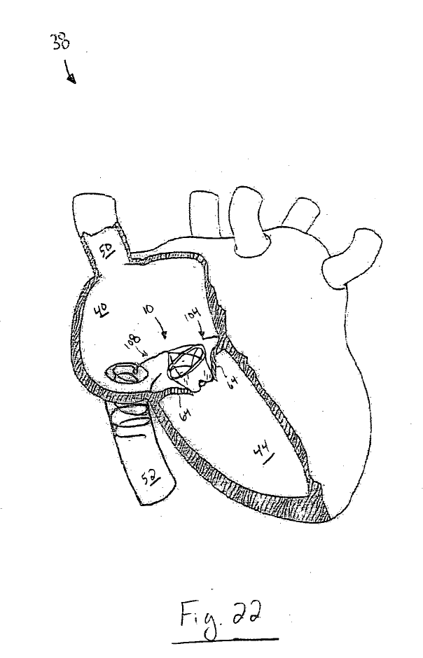

After the apparatus 10 is freely suspended in the diseased tricuspid valve 104, the positioning wire 102 is disconnected from the apparatus and, along with the guidewire 84, may be withdrawn from the patient's vasculature. With the valve member 12 appropriately positioned in the regurgitant tricuspid valve orifice 106, blood may flow normally through and around the valve member during diastole (FIG. 21). Then, during systole, at least one leaflet 64 of the diseased tricuspid valve 104 can coapt with the surface of the valve member 12 as shown in FIG. 22. Consequently, the valve leaflets 64 can abut the valve member 12 and buttress the diseased tricuspid valve 104 so that the regurgitant blood flow through the diseased tricuspid valve is substantially reduced or eliminated during systole.

From the above description of the invention, those skilled in the art will perceive improvements, changes and modifications. The apparatus 10 may be delivered to the heart 38 via a non-percutaneous method by, for example, obtaining open-chest access to a diseased cardiac valve 108. Such improvements, changes and modifications within the skill of the art are intended to be covered by the appended claims.

Having described the invention, we claim:

1. An apparatus for treating regurgitation of blood through a diseased valve having at least one leaflet, said apparatus comprising:
a valve member comprising a support structure with a diameter and at least one valvular leaflet attached to said support structure, said valve member being dimensioned so that at least one leaflet of the diseased valve abuts at least one surface of said valve member to mitigate regurgitation of blood through the diseased valve; and
a suspending mechanism operatively coupled to said valve member, said suspending mechanism configured so that said valve member is freely suspended within the diseased valve.

2. The apparatus of claim 1, wherein said suspending mechanism is operatively securable to a vascular wall surrounding the diseased valve, said suspending mechanism positioned so that said valve member is freely suspended by said suspending mechanism within the diseased valve and at least a portion of said valve member is positioned adjacent to the at least one leaflet of the diseased valve, said portion contacting at least one surface of the at least one leaflet.

3. The apparatus of claim 1, wherein at least a portion of said valve member is configured to be positioned between the valve leaflets.

4. The apparatus of claim 1, wherein at least a portion of said valve member is configured to be positioned below the free ends of the valve leaflets.

5. The apparatus of claim 1, wherein at least a portion of said valve member is configured to be positioned approximately at a level of the annulus of the valve.

6. The apparatus of claim 1, wherein said valve member comprises a mechanical valve.

7. The apparatus of claim 1, wherein said valve member comprises a bioprosthetic valve.

8. The apparatus of claim 1, wherein said support structure is collapsible to a smaller diameter.

9. The apparatus of claim 1, wherein said support structure further comprises an adjustable sizing member for adjusting the diameter of said valve member within the diseased valve.

10. The apparatus of claim 9, wherein said adjustable sizing member further comprises an inflatable balloon encircling at least a portion of said support structure.

11. The apparatus of claim 1, wherein the diseased valve is located in the arterial vasculature.

12. The apparatus of claim 1, wherein the diseased valve is located in the venous vasculature.

13. The apparatus of claim 1, wherein the diseased valve is a heart valve.

14. A method for treating regurgitation of blood through a diseased valve, said method comprising the steps of:
providing an apparatus comprising a valve member and a suspending mechanism operatively coupled to the valve member, the valve member comprising a support structure with a diameter and at least one valvular leaflet attached to the support structure;
positioning a balloon in the diseased valve to determine the size and shape of the diseased valve;
selecting a valve member having a size and shape that corresponds to the size and shape of the diseased valve so that at least one leaflet of the diseased valve coapts with the valve member;
introducing the apparatus into a patient's vasculature; and
positioning the apparatus in the diseased valve.

15. The method of claim 14, wherein the balloon comprises a first layer and second layer.

16. The method of claim 14, wherein the second layer encapsulates at least one portion of the first layer.

17. The method of claim 16, wherein the at least one portion of the second layer has a non-uniform thickness.

18. The method of claim 14, wherein said step of positioning the balloon in the diseased valve further comprises the steps of:
positioning the balloon in a deflated configuration in a regurgitant orifice of the diseased valve;
inflating the balloon so that blood flow through the regurgitant orifice is substantially hindered; and
measuring the geometry of the balloon in at least one of a plurality of dimensions.

19. The method of claim 14, wherein said step of positioning the apparatus in the diseased valve comprises the steps of:
extending the apparatus into a portion of the diseased valve; and
suspending the apparatus in the diseased valve so that at least one leaflet of the diseased valve coapts with the valve member to substantially hinder regurgitant bloodflow through the valve.

20. The method of claim 14, wherein said step of positioning the apparatus in the diseased valve further comprises the step of adjusting the diameter of the valve member by altering the diameter of the support structure.
